(19) United States
(12) Patent Application Publication
(10) Pub. No.: US 2022/0183836 A1
(43) Pub. Date: Jun. 16, 2022

(54) REPLACEMENT HEART VALVE DELIVERY DEVICE

(71) Applicant: BOSTON SCIENTIFIC SCIMED, INC., MAPLE GROVE, MN (US)

(72) Inventors: Declan Loughnane, Galway (IE); Tim O'Connor, Galway (IE); Takashi H. Ino, San Jose, CA (US)

(73) Assignee: BOSTON SCIENTIFIC SCIMED, INC., MAPLE GROVE, MN (US)

(21) Appl. No.: 17/602,965

(22) PCT Filed: Apr. 9, 2020
(86) PCT No.: PCT/US2020/027503

§ 371 Date: Oct. 11, 2021

Related U.S. Application Data
(60) Provisional application No. 62/833,053, filed on Apr. 12, 2019.

(51) Int. Cl. A61F 2/24

(52) U.S. Cl. CPC A61F 2/2436

(57) ABSTRACT

A medical device system may include a delivery device including an outer sheath and an inner shaft having a coupler fixed to a distal end of the inner shaft; and a replacement heart valve implant releasably attached to the coupler. The replacement heart valve implant may include an expandable anchor member defining a central longitudinal axis; and a locking mechanism attached to the expandable anchor member, the locking mechanism being configured to engage with the coupler. The delivery device may include a collar configured to releasably secure the coupler to the locking mechanism. The collar may include a stop element configured to selectively prevent disengagement of the collar from the locking mechanism

### CROSS-REFERENCE TO RELATED APPLICATIONS

This application claims the benefit of priority of U.S. Provisional Application No. 62/833,053 filed Apr. 12, 2019, as well as PCT Application No. PCT/US2020/027503, filed Apr. 9, 2020. The entirety of these disclosures of which is hereby incorporated by reference.

### TECHNICAL FIELD

The present disclosure pertains to medical devices, and methods for manufacturing and/or using medical devices. More particularly, the present disclosure pertains to a delivery device for a replacement heart valve implant.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In a first aspect, a medical device system may comprise a delivery device including an outer sheath and an inner shaft having a coupler fixed to a distal end of the inner shaft; and a replacement heart valve implant releasably attached to the coupler. The replacement heart valve implant may include an expandable anchor member defining a central longitudinal axis; and a locking mechanism attached to the expandable anchor member, the locking mechanism being configured to engage with the coupler. The delivery device may include a collar configured to releasably secure the coupler to the locking mechanism. The collar may include a stop element configured to selectively prevent disengagement of the collar from the locking mechanism.

In addition or alternatively, and in a second aspect, the collar is slidable over the coupler between a first position and a second position. In the first position, the collar may secure the coupler to the locking mechanism, and in the second position the coupler may be disengageable from the locking mechanism. The delivery device may further include a locking rod extending through at least a portion of the collar, the locking rod being actuatable between a latch position and a release position. In the latch position the locking rod may engage the stop element.

In addition or alternatively, and in a third aspect, the stop element includes a leg portion deflectable in a circumferential direction relative to the central longitudinal axis.

In addition or alternatively, and in a fourth aspect, the leg portion includes a longitudinally-oriented slot configured to receive at least a portion of the coupler when the locking rod is in the latch position.

In addition or alternatively, and in a fifth aspect, the leg portion extends longitudinally along a side of the collar.

In addition or alternatively, and in a sixth aspect, the second position is proximal of the first position.

In addition or alternatively, and in a seventh aspect, the release position is proximal of the latch position.

In addition or alternatively, and in an eighth aspect, the locking rod is actuatable from the latch position to the release position and the locking rod is not actuatable from the release position to the latch position.

In addition or alternatively, and in a ninth aspect, a medical device system may comprise a delivery device including an outer sheath and an inner shaft having a coupler fixed to a distal end of the inner shaft; and a replacement heart valve implant releasably attached to the coupler. The replacement heart valve implant may include an expandable anchor member defining a central longitudinal axis; and a locking mechanism attached to the expandable anchor member, the locking mechanism being configured to engage with the coupler. The delivery device may include a collar slidable over the coupler between a first position and a second position, the collar being configured to releasably secure the coupler to the locking mechanism. In the first position the collar may secure the coupler to the locking mechanism, and in the second position the coupler may be disengageable from the locking mechanism. The delivery device may further include a locking rod extending through at least a portion of the collar, the locking rod being actuatable between a latch position and a release position. In the latch position the locking rod secures the collar in the first position.

In addition or alternatively, and in a tenth aspect, in the latch position a portion of the locking rod distal of a proximal end of the collar is disposed outside of at least a portion of the collar.

In addition or alternatively, and in an eleventh aspect, in the latch position the locking rod deflects a leg portion of the collar inward toward the coupler.

In addition or alternatively, and in a twelfth aspect, in the latch position the locking rod deflects a proximal end of the leg portion of the collar into a recess formed in the coupler.

In addition or alternatively, and in a thirteenth aspect, when the locking rod is in the release position the collar is permitted to slide from the first position to the second position.

In addition or alternatively, and in a fourteenth aspect, the delivery device includes an actuator element extending through the collar and releasably engaged with the locking mechanism.

In addition or alternatively, and in a fifteenth aspect, when the locking rod is in the release position proximal translation of the actuator element shifts the collar from the first position to the second position.

In addition or alternatively, and in a sixteenth aspect, a medical device system may comprise a delivery device including an outer sheath and an inner shaft having a coupler fixed to a distal end of the inner shaft, the outer sheath and the inner shaft extending distally from a handle; and a replacement heart valve implant releasably attached to the coupler. The replacement heart valve implant may include an expandable anchor member defining a central longitudinal axis; and a locking mechanism attached to the expandable anchor member, the locking mechanism being configured to engage with the coupler. The locking mechanism may include a buckle member fixedly attached to the expandable anchor member, and a post member coupled to the expandable anchor member distal of the buckle member. The delivery device may include a collar slidable over the coupler between a first position and a second position. In the first position the collar secures the coupler to the buckle member, and in the second position the coupler is disengageable from the buckle member. The delivery device may further include a locking rod extending through at least a portion of the collar, the locking rod being actuatable between a latch position and a release position. In the latch position the locking rod deflects a leg portion of the collar into a recess formed in the coupler, thereby preventing the collar from sliding from the first position to the second position.

In addition or alternatively, and in a seventeenth aspect, the leg portion is self-biased to be in a neutral configuration.

In addition or alternatively, and in an eighteenth aspect, when the leg portion is deflected into the recess, the leg portion is self-biased outwardly from the coupler.

In addition or alternatively, and in a nineteenth aspect, the recess defines an engagement surface facing at least partially in a distal direction. When the locking rod is in the latch position, interference between the engagement surface and a proximal end of the leg portion prevents the collar from sliding from the first position to the second position.

In addition or alternatively, and in a twentieth aspect, at the collar, the locking rod is circumferentially offset from the coupler with respect to the central longitudinal axis.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example medical device system;

FIG. 2 is a perspective view of selected elements of the medical device system in a deployed configuration, a first position, and/or a latch position;

FIG. 3 illustrates selected elements of an example replacement heart valve implant in a delivery configuration, the first position, and/or the latch position;

FIG. 4 is a top view of selected elements of the example replacement heart valve implant of FIG. 3;

FIG. 5 illustrates selected elements of an example replacement heart valve implant in a deployed configuration, the first position, and/or the latch position;

FIG. 6 illustrates an example medical device system in the deployed configuration;

FIG. 7 is a perspective view of selected elements of the medical device system in the deployed configuration, the first position, and/or a release position;

FIG. 8 illustrates selected elements of an example replacement heart valve implant in the deployed configuration, a second position, and/or the release position;

FIG. 9 illustrates selected elements of an example replacement heart valve implant in a released configuration, the second position, and/or the release position; and

FIG. 10 is a perspective view of selected elements of the medical device system in the released configuration, the second position, and/or the release position.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein is a medical device system that may be used for delivering a medical device to a portion of the cardiovascular system in order to diagnose, treat, and/or repair the system. At least some of the medical devices and/or systems disclosed herein may be used to deliver and implant a replacement heart valve implant (e.g., a replacement aortic valve, replacement mitral valve, etc.). In addition, the medical device system may deliver the replacement heart valve implant percutaneously and, thus, may be much less invasive to the patient. The device and/or system disclosed herein may also provide other desirable features and/or benefits as described below.

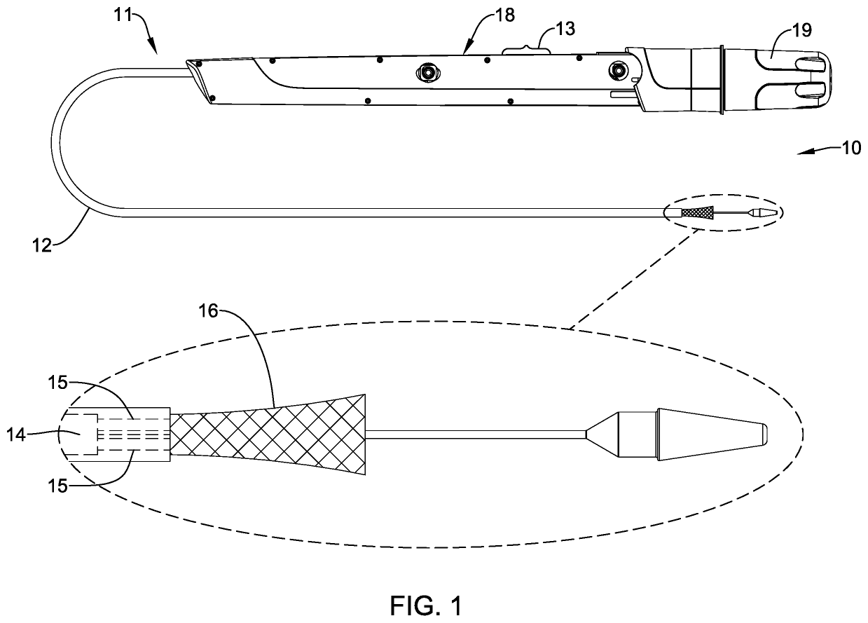

The figures illustrate selected components and/or arrangements of a medical device system 10, shown schematically in FIG. 1 for example. It should be noted that in any given figure, some features of the medical device system 10 may not be shown, or may be shown schematically, for simplicity. Additional details regarding some of the components of the medical device system 10 may be illustrated in other figures in greater detail. A medical device system 10 may be used to deliver and/or deploy a variety of medical devices and/or implants to one or more locations within the anatomy. In at least some embodiments, the medical device system 10 may include a delivery device 11 (e.g., a replacement heart valve delivery system) that can be used for percutaneous delivery of a replacement heart valve implant 16 (e.g., a replacement mitral valve, a replacement aortic valve, etc.) to an area of interest in the anatomy, such as a native heart valve. This, however, is not intended to be limiting as the medical device system 10 and/or the delivery device 11 may also be used for other interventions including valve repair, valvuloplasty, and the like, or other similar interventions.

FIG. 1 illustrates the medical device system 10 including the replacement heart valve implant 16 configured to be disposed within the area of interest, such as a native heart valve (e.g., a mitral valve, an aortic valve, etc.), wherein the replacement heart valve implant 16 may be disposed within a lumen of the delivery device 11 in a delivery configuration for delivery to the area of interest, where the replacement heart valve implant 16 may be shifted to a deployed configuration. In some embodiments, the delivery device 11 may include an outer sheath 12 having a lumen extending from a proximal portion and/or proximal end of the outer sheath 12 to a distal end of the outer sheath 12. The replacement heart valve implant 16 may be disposed within the lumen of the outer sheath 12 proximate the distal end of the outer sheath 12 in the delivery configuration. In some embodiments, the delivery device 11 may include a handle 18 disposed proximate and/or at the proximal end of the outer sheath 12. The handle 18 may have an outer shell and an interior space. In some embodiments, the handle 18 may have a control knob 19 rotatable relative to the outer shell of the handle 18. In some embodiments, the handle 18 may optionally include a slider 13 axially and/or longitudinally slidable relative to the outer shell of the handle 18.

The delivery device 11 may include an inner shaft 14 disposed within the lumen of the outer sheath 12 and/or slidable with respect to the outer sheath 12 within the lumen of the outer sheath 12. In some embodiments, the handle 18 may be disposed proximate and/or at a proximal end of the inner shaft 14. In some embodiments, the inner shaft 14 may be a tubular structure having one or more lumens extending therethrough, the inner shaft 14 may be a solid shaft, or the inner shaft 14 may be a combination thereof. In some embodiments, the proximal end of the outer sheath 12 and the proximal end of the inner shaft 14 may each be operably connected, fixed, and/or secured to an axial translation mechanism disposed within the outer shell of the handle 18. The axial translation mechanism may be configured to move and/or translate the outer sheath 12 relative to the inner shaft 14.

In some embodiments, the delivery device 11 may include an actuator element 15 releasably connecting the replacement heart valve implant 16 to the handle 18. For example, the actuator element 15 may extend from the handle 18 to the replacement heart valve implant 16, the replacement heart valve implant 16 being disposed at a distal end of the lumen of the outer sheath 12. The actuator element 15 may extend distally from the inner shaft 14 to the replacement heart valve implant 16. In some embodiments, the actuator element 15 may be slidably disposed within and/or may extend slidably through the inner shaft 14.

The handle 18 and/or the actuator element 15 may be configured to manipulate the position of the outer sheath 12 relative to the inner shaft 14 and/or aid in the deployment of the replacement heart valve implant 16. For example, the inner shaft 14 and/or the actuator element 15 may be used to move the replacement heart valve implant 16 with respect to the outer sheath 12 of the delivery device 11. In some embodiments, the inner shaft 14 and/or the actuator element 15 may be advanced distally within the lumen of the outer sheath 12 to push the replacement heart valve implant 16 out the distal end of the outer sheath 12 and/or the delivery device 11 to deploy the replacement heart valve implant 16 within the area of interest (e.g., the native heart valve, etc.). In some embodiments, the inner shaft 14 and/or the actuator element 15 may be held in a fixed position relative to the replacement heart valve implant 16 and the outer sheath 12 may be withdrawn proximally relative to the inner shaft 14, the actuator element 15, and/or the replacement heart valve implant 16 to deploy the replacement heart valve implant 16 within the area of interest (e.g., the native heart valve, etc.). Some examples of suitable but non-limiting materials for the medical device system 10, the delivery device 11, the outer sheath 12, the slider 13, the inner shaft 14, the actuator element 15, the handle 18, the control knob 19, and/or components or elements thereof, are described below.

In some embodiments, the delivery device 11 may include a nose cone disposed at a distal end of a guidewire extension tube, wherein the guidewire extension tube may extend distally from the inner shaft 14 and/or the outer sheath 12. In at least some embodiments, the nose cone may be designed to have an atraumatic shape and/or may include a ridge or ledge that is configured to abut a distal end of the outer sheath 12 during delivery of the replacement heart valve implant 16.

In use, the medical device system 10 and/or the delivery device 11 may be advanced percutaneously through the vasculature to the area of interest. For example, the medical device system 10 and/or the delivery device 11 may be advanced over a guidewire through the vasculature and across the aortic arch to a defective heart valve (e.g., aortic valve, mitral valve, etc.). The guidewire may be slidably disposed within and/or may slidably extend through the nose cone, the guidewire extension tube, the inner shaft 14, the outer sheath 12, and/or the handle 18. Alternative approaches to treat a defective heart valve are also contemplated with the medical device system 10 and/or the delivery device 11. During delivery, the replacement heart valve implant 16 may be generally disposed in an elongated and low profile “delivery” configuration within the lumen of the outer sheath 12. Once positioned, the outer sheath 12 may be retracted relative to the replacement heart valve implant 16 and/or the inner shaft 14 to expose the replacement heart valve implant 16. In at least some embodiments, the replacement heart valve implant 16 may be disposed in an “everted” configuration or a partially-everted configuration while disposed within the lumen of the outer sheath 12 and/or immediately upon exposure after retracting the outer sheath 12. In some embodiments, the replacement heart valve implant 16 may be everted in the “delivery” configuration. The “everted” configuration may involve at least a portion of the valve leaflets (discussed below) of the replacement heart valve implant 16 being disposed outside of the expandable anchor member (discussed below) of the replacement heart valve implant 16 during delivery, thereby permitting a smaller radial profile of the replacement heart valve implant 16 and the use of a smaller overall profile of the outer sheath 12, the delivery device 11, and/or the medical device system 10. In some embodiments, the “delivery” configuration and the “everted” configuration may be substantially similar and/or may be used interchangeably herein.

The replacement heart valve implant 16 may be actuated using the handle 18, the control knob 19, and/or the actuator element 15 in order to translate the replacement heart valve implant 16 into a radially expanded and larger profile “deployed” configuration suitable for implantation within the anatomy at the area of interest or the target location. The replacement heart valve implant 16 may be actuated from the “delivery” configuration to the “deployed” configuration. After verifying placement of the replacement heart valve implant 16 using a suitable imaging technique, the handle 18, the control knob 19, and/or the slider 13 may be subsequently actuated to shift the replacement heart valve implant 16 into a “released” configuration, as discussed herein. When the replacement heart valve implant 16 is suitably deployed and released within the area of interest, the outer sheath 12 and/or the delivery device 11 can be removed from the vasculature. In at least some interventions, the replacement heart valve implant 16 may be deployed within the native heart valve (e.g., the native heart valve is left in place and not excised). Alternatively, the native heart valve may be removed and the replacement heart valve implant 16 may be deployed in its place as a replacement.

Disposed within a first lumen of the inner shaft 14 may be the actuator element 15, which may be used to actuate and/or translate (e.g., expand and/or elongate) the replacement heart valve implant 16 between the “delivery” configuration and the “deployed” configuration. In some embodiments, the actuator element 15 may include or comprise a plurality of actuator elements 15, two actuator elements 15, three actuator elements 15, four actuator elements 15, or another suitable or desired number of actuator elements 15. In some embodiments, each actuator element 15 may be disposed within a separate lumen of the inner shaft 14. For the purpose of illustration only, the medical device system 10, the delivery device 11, and the replacement heart valve implant 16 are shown with three actuator elements 15. In such an example, the three actuator elements 15 may be disposed within three separate lumens (e.g., a first lumen, a second lumen, and a third lumen) of the inner shaft 14, although such a configuration is not required.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the actuator element” may be equally referred to all instances and quantities beyond one of “the at least one actuator element” or “the plurality of actuator elements”.

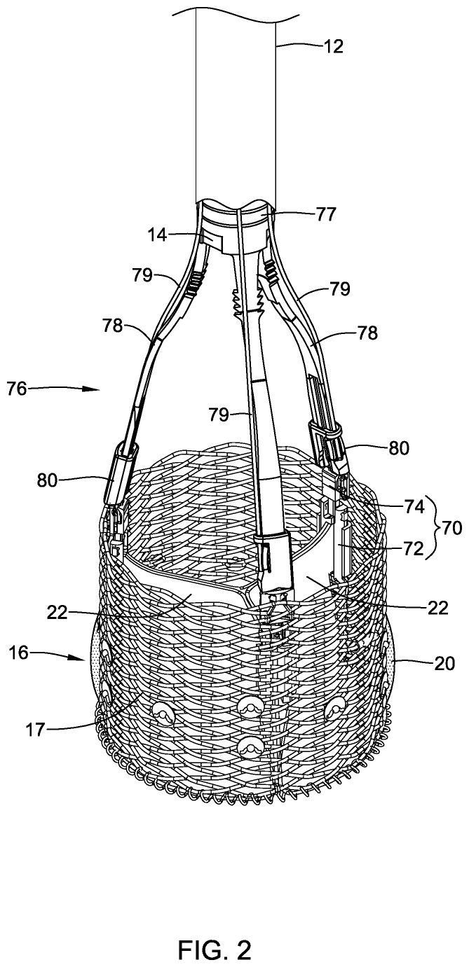

FIG. 2 illustrates some selected components of the medical device system 10, the delivery device 11, and/or the replacement heart valve implant 16, shown in the “deployed” configuration. The replacement heart valve implant 16 may include an expandable anchor member 17 that is reversibly actuatable between the elongated “delivery” configuration and the radially expanded and/or axially shortened “deployed” configuration. In some embodiments, the expandable anchor member 17 may be tubular and defines a lumen extending coaxially along a central longitudinal axis from a distal or inflow end of the expandable anchor member 17 and/or the replacement heart valve implant 16 to a proximal or outflow end of the expandable anchor member 17 and/or the replacement heart valve implant 16.

In some embodiments, the expandable anchor member 17 may comprise an expandable stent structure and/or framework. In some embodiments, the expandable anchor member 17 may comprise a self-expanding braided and/or woven mesh structure made up of one or more filaments disposed and/or interwoven circumferentially about the lumen of the expandable anchor member 17 and/or the replacement heart valve implant 16. Non-self-expanding, mechanically-expandable, and/or assisted self-expanding expandable anchor members are also contemplated. In at least some embodiments, the expandable anchor member 17 may be formed as a unitary structure (e.g., formed from a single filament or strand of wire, cut from a single tubular member, etc.). In some embodiments, the expandable anchor member 17 may define a generally cylindrical outer surface in the deployed configuration. Other configurations are also possible—a cross-section defining a generally elliptical outer surface, for example. Some examples of suitable but non-limiting materials for the replacement heart valve implant 16, the expandable anchor member 17, and/or components or elements thereof, are described below.

Also shown in FIG. 2, but omitted from several other figures in the interest of clarity, the replacement heart valve implant 16 may include a plurality of valve leaflets 22 disposed within the lumen of the replacement heart valve implant 16 and/or the expandable anchor member 17. In some embodiments, the plurality of valve leaflets 22 may be attached and/or secured to the expandable anchor member 17 at a plurality of locations within the lumen of the replacement heart valve implant 16 and/or the expandable anchor member 17. In some embodiments, the plurality of valve leaflets 22 may be attached and/or secured to the expandable anchor member 17 using sutures, adhesives, or other suitable means.

In some embodiments, the plurality of valve leaflets 22 may include or comprise two leaflets, three leaflets, four leaflets, etc. as desired. For example, the plurality of valve leaflets 22 may comprise a first valve leaflet, a second valve leaflet, a third valve leaflet, etc. and may be referred to collectively as the plurality of valve leaflets 22. The plurality of valve leaflets 22 of the replacement heart valve implant 16 may be configured to move between an open configuration permitting antegrade fluid flow through the replacement heart valve implant 16 and/or the lumen of the replacement heart valve implant 16 and/or the expandable anchor member 17, and a closed configuration preventing retrograde fluid flow through the replacement heart valve implant 16 and/or the lumen of the replacement heart valve implant 16 and/or the expandable anchor member 17. The plurality of valve leaflets 22 may each have a free edge, wherein the free edges of the plurality of valve leaflets 22 coapt within the replacement heart valve implant 16, the expandable anchor member 17, and/or the lumen extending through the replacement heart valve implant 16 and/or the expandable anchor member 17 in the closed configuration. Some examples of suitable but non-limiting materials for the plurality of valve leaflets 22 may include bovine pericardial, polymeric materials, or other suitably flexible biocompatible materials.

The replacement heart valve implant 16 may include a replacement heart valve commissure assembly disposed within the lumen of the replacement heart valve implant 16 and/or the expandable anchor member 17. In some embodiments, the replacement heart valve implant 16 may include more than one replacement heart valve commissure assembly. For example, each adjacent pair of valve leaflets 22 may form and/or define one replacement heart valve commissure assembly. Therefore, the number of replacement heart valve commissure assemblies may be directly related to the number of valve leaflets 22 (e.g., three valve leaflets form and/or define three replacement heart valve commissure assemblies, two valve leaflets form and/or define two replacement heart valve commissure assemblies, etc.).

In some embodiments, the replacement heart valve implant 16 and/or the replacement heart valve commissure assembly may include a locking mechanism 70 configured to lock the expandable anchor member 17 in the “deployed” configuration, as seen in FIG. 2. In some embodiments, the replacement heart valve implant 16 may include or comprise a plurality of locking mechanisms (e.g., two locking mechanisms, three locking mechanisms, etc.). In some embodiments, each replacement heart valve commissure assembly may correspond to and/or include at least one corresponding locking mechanism 70. Each locking mechanism 70 may include a first locking portion or a post member 72 coupled to the expandable anchor member 17 and configured to engage with a second locking portion or a buckle member 74 fixedly attached to the expandable anchor member 17, as will be described in more detail below.

In some embodiments, the actuator element 15 may be configured to releasably engage the locking mechanism 70 and/or reversibly actuate the expandable anchor member 17 and/or the replacement heart valve implant 16 between the “delivery” configuration and the “deployed” configuration while the actuator element 15 is engaged with the locking mechanism 70. In some embodiments, one actuator element 15 may correspond to, engage with, and/or actuate one locking mechanism 70. In some embodiments, one actuator element 15 may correspond to, engage with, and/or actuate more than one locking mechanism 70. Other configurations are also contemplated.

In some embodiments, the actuator element 15 may include a proximal end and a distal end. In use, the proximal end may be operatively connected to the handle 18 and/or the control knob 19, and/or manipulated or otherwise actuated by a user using the handle 18 and/or the control knob 19, to reversibly shift the replacement heart valve implant 16 between the “delivery” configuration and the “deployed” configuration. For example, the control knob 19, rotatable relative to the outer shell of the handle, may be actuatable and/or rotatable to manipulate or otherwise actuate the actuator element 15, the outer sheath 12, and/or the inner shaft 14. In some embodiments, the actuator element 15 may be axially translatable relative to the first locking portion or post member 72 and/or the second locking portion or buckle member 74 of the replacement heart valve implant 16.

In some embodiments, the proximal end of the actuator element 15 (each actuator element 15, etc.) may be operatively connected to a central shaft extending distally from the handle 18 within the inner shaft 14. The central shaft may be actuated and/or translated by the handle 18, the control knob 19, and/or a mechanism disposed within the handle 18 responsive to the control knob 19. In some embodiments, the actuator element 15 (each actuator element 15, etc.) may extend distally from the handle 18 within the inner shaft 14.

In some embodiments, the replacement heart valve implant 16 may include a seal member 20 (shown partially cutaway in FIG. 2) disposed on and/or around at least a portion of the outer surface of the expandable anchor member 17. In some embodiments, the seal member 20 may be attached and/or secured to the distal or inflow end of the expandable anchor member 17 and/or the replacement heart valve implant 16, and/or the seal member 20 may be attached and/or secured to the plurality of valve leaflets 22 proximate the distal or inflow end of the expandable anchor member 17 and/or the replacement heart valve implant 16. The seal member 20 may be sufficiently flexible and/or pliable to conform to and/or around native valve leaflets and/or the native heart valve in the deployed configuration, thereby sealing an exterior of the replacement heart valve implant 16 and/or the expandable anchor member 17 within and/or against the native heart valve and/or the native valve leaflets and preventing leakage around the replacement heart valve implant 16 and/or the expandable anchor member 17.

In some embodiments, the seal member 20 may include one or more layers of polymeric material. Some suitable polymeric materials may include, but are not necessarily limited to, polycarbonate, polyurethane, polyamide, polyether block amide, polyethylene, polyethylene terephthalate, polypropylene, polyvinylchloride, polytetrafluoroethylene, polysulfone, and copolymers, blends, mixtures or combinations thereof. Other suitable polymeric materials are also contemplated, some of which are discussed below.

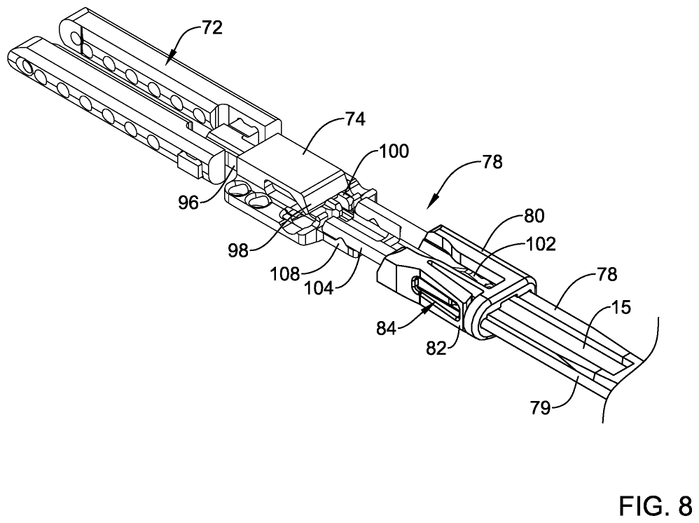

During delivery, the replacement heart valve implant 16 and/or the expandable anchor member 17 may be secured at the distal end of the inner shaft 14 by a coupler 76 coupled with a projecting portion 108 (e.g., FIG. 8) at a proximal end of the second locking portion or buckle member 74 and being held in place with a collar 80 disposed over the connection. As such, the inner shaft 14 of the delivery device 11 may include the coupler 76 fixed to the distal end of the inner shaft 14, and the replacement heart valve implant 16 may be releasably attached to the coupler 76. The locking mechanism 70 and/or the second locking portion or buckle member 74 may be configured to engage with the coupler 76.

The coupler 76 may include a proximal ring 77 fixedly attached to the distal end of the inner shaft 14 and a plurality of fingers 78 extending distally from the proximal ring 77. In at least some embodiments, the plurality of fingers 78 may be integrally formed with the proximal ring 77 as a single, unitary structure. In some embodiments, each finger 78 of the coupler 76 may include a collar 80 slidably disposed on and/or about its respective finger 78 and the projecting portion 108 of its respective second locking portion or buckle member 74. The collar 80 may be configured to releasably secure the coupler 76 to the locking mechanism 70 and/or the second locking portion or buckle member 74. The collar 80 may be slidable over the finger 78 of the coupler 76 between a first position and a second position. In the first position, the collar 80 may secure the coupler 76 to the locking mechanism 70 and/or the second locking portion or buckle member 74. In the second position, the coupler 76 may be disengageable from the locking mechanism 70 and/or the second locking portion or buckle member 74.

The collar 80 may be configured to maintain engagement of its respective finger 78 with its respective locking mechanism 70 and/or second locking portion or buckle member 74. As there may be a plurality of second locking portions or buckle members in the plurality of locking mechanisms, there may likewise be a plurality of collars securing the replacement heart valve implant 16 and/or the expandable anchor member 17 to the coupler 76. The plurality of fingers 78 may be releasably coupled to the locking mechanism 70 and/or the second locking portion or buckle members 74 of the plurality of locking mechanisms by the plurality of collars. Some suitable but non-limiting materials for the coupler 76, the proximal ring 77, the plurality of fingers 78, and/or the collar 80, for example shape memory materials, metallic materials, and/or polymeric materials, are described below.

The medical device system 10 and/or the delivery device 11 may include a locking rod 79 associated with and/or engageable with each collar 80. In a medical device system 10 and/or a delivery device 11 having a plurality of fingers 78 and/or a plurality of collars 80, the medical device system 10 and/or the delivery device 11 may also have a plurality of locking rods 79. For example, if the medical device system 10 and/or the delivery device 11 has three fingers 78 and three collars 80, the medical device system 10 and/or the delivery device 11 may also have three locking rods 79, as shown in FIG. 2. The locking rod 79 may extend through at least a portion of the collar 80. The locking rod 79 may be actuatable between a latch position and a release position. The locking rod 79 may be operatively connected to the handle 18, the control knob 19, a mechanism disposed within the handle 18 responsive to the control knob 19, and/or the slider 13. In one example, the locking rod 79 may be operatively connected to the same feature or structure as the actuator element 15. In another example, the locking rod 79 may be operatively connected to the slider 13, which may be operable independently of the handle 18, the control knob 19, and/or the mechanism disposed within the handle 18 responsive to the control knob 19. In some embodiments, the locking rod 79 may be axially translatable between the latch position and the release position. Some suitable but non-limiting materials for the locking rod 79, for example shape memory materials, metallic materials, and/or polymeric materials, are described below.

In some embodiments, a tubular guide member (not shown) may be disposed over a distal portion of each of the plurality of fingers 78 proximal of the collar 80, and may serve to keep the distal portion of each of the plurality of fingers 78 of the coupler 76 associated with its respective actuator element 15 extending adjacent to (and axially slidable relative to) the plurality of fingers 78 of the coupler 76. In some embodiments, the tubular guide member may also be disposed over the locking rod 79 (e.g., the locking rod 79 may extend through the tubular guide member) and function to keep the locking rod 79 associated with its respective finger 78.

In use, after the replacement heart valve implant 16 and/or the expandable anchor member 17 is advanced within the anatomy to the area of interest, the handle 18, the control knob 19, a mechanism disposed within the handle 18 responsive to the control knob 19, and/or the actuator element 15 can be used to actuate the replacement heart valve implant 16 and/or the expandable anchor member 17 from the “delivery” configuration to the “deployed” configuration by proximally retracting the actuator element 15 relative to the second locking portion or buckle member 74 and/or the expandable anchor member 17, thereby pulling the first locking portion or post member 72 into engagement with the second locking portion or buckle member 74, as discussed herein.

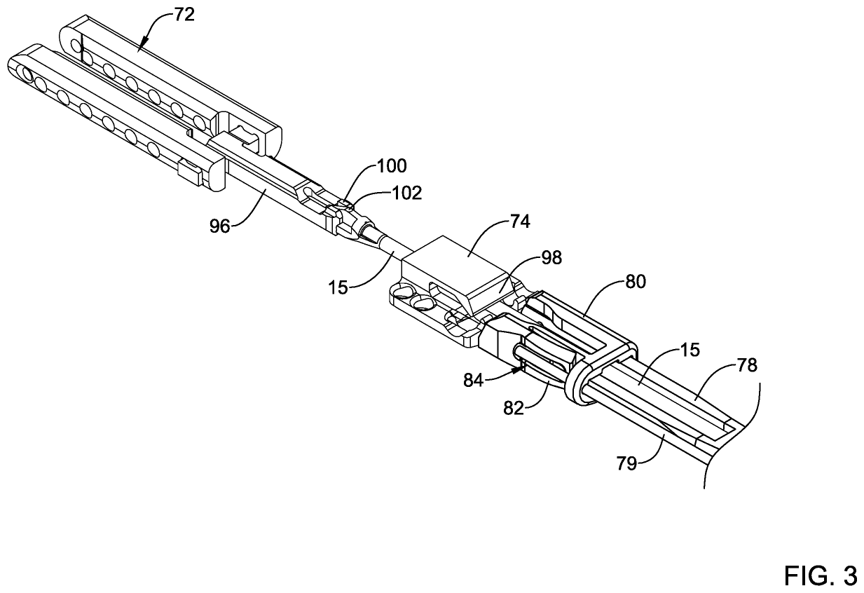

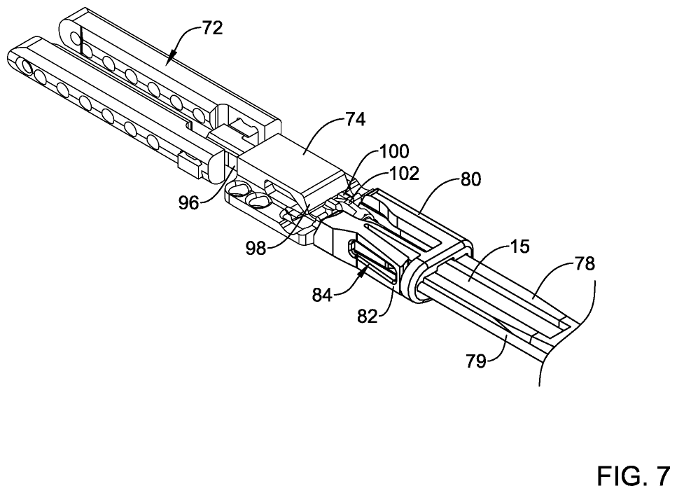

FIGS. 3-5 (as well as FIGS. 7-9) illustrate selected components and/or details of an example locking mechanism 70 and/or certain components related to the locking mechanism 70, and the general operation of those components. For simplicity and clarity purposes, only one finger 78, only one locking rod 79, only one collar 80, only one actuator element 15, only one first locking portion or post member 72, and only one second locking portion or buckle member 74 are shown and discussed (the whole replacement heart valve implant 16 and/or the expandable anchor member 17 is not shown to facilitate understanding of the locking mechanism(s) 70 and related elements). However, it will be understood that the following discussion may apply equally to any and/or all of the components for which there are more than one within the replacement heart valve implant 16 (i.e., the actuator elements 15, the second locking portions or buckle members 74, the first locking portions or post members 72, the locking rods 79, the collars 80, etc.), the delivery device 11, and/or the medical device system 10.

In some embodiments, the actuator element 15 (e.g., each actuator element 15, etc.) includes an elongated rod having a distal portion and a ramp 102 (e.g., FIG. 3) extending longitudinally and/or radially outward from the actuator element 15 such that the ramp 102 has a greater outer extent than the elongated rod. The ramp 102 may be positioned proximate to and/or at a proximal end of the distal portion of the actuator element 15.

In some embodiments, the distal portion of the actuator element 15 may be aligned with and/or releasably coupled to the first locking portion or post member 72. In some embodiments, the distal portion of the actuator element 15 may be slidably received within a longitudinally-oriented passageway of the first locking portion or post member 72, as discussed below. The handle 18, the control knob 19, and/or the mechanism disposed within the handle 18 responsive to the control knob 19 may be configured to actuate and/or translate the actuator element 15 (e.g., each actuator element 15, etc.) relative to the outer sheath 12, the replacement heart valve implant 16, the corresponding locking mechanism(s) 70 (e.g., the plurality of locking mechanisms 70, etc.), and/or the first locking portion or post member 72 in the “delivery” and/or “deployed” configuration. The actuator element 15 may be axially and/or slidably translatable through and/or relative to the collar 80 and/or the second locking portion or buckle member 74.

In some embodiments, the actuator element 15 and/or the elongated rod may be generally round, oblong, ovoid, rectangular, polygonal (e.g., two-sided, three-sided, four-sided, five-sided, six-sided, etc.) and/or combinations thereof in shape. Other shapes, both regular and irregular, are also contemplated. In some embodiments, the actuator element 15 may be formed from a single piece of wire, round stock, or other suitable material, as discussed herein. In some embodiments, the actuator element 15 may be formed by further processing the single piece of wire, round stock, or other suitable material, such as by machining, stamping, laser cutting, etc. Some suitable but non-limiting materials for the actuator element 15, the elongated rod, the distal portion, and/or the ramp, for example metallic materials or polymeric materials, are described below.

In some embodiments, the first locking portion or post member 72 and the second locking portion or buckle member 74 may be longitudinally movable relative to each other along an inner surface of the expandable anchor member 17 in the “delivery” configuration and/or the “deployed” configuration. In some embodiments, the first locking portion or post member 72 may be non-releasably coupled to a distal portion and/or proximate the distal or upstream end of the expandable anchor member 17 along the inner surface of the expandable anchor member 17. In some embodiments, the second locking portion or buckle member 74 may be fixedly attached to a proximal portion and/or proximate the proximal or downstream end of the expandable anchor member 17 against the inner surface of the expandable anchor member 17. The first locking portion or post member 72 may be coupled to the expandable anchor member 17 distal of the second locking portion or buckle member 74. The second locking portion or buckle member 74 may be configured to slidably receive at least a portion of the first locking portion or post member 72 therein. Additional discussion regarding the relative motion of these elements is provided below.

In at least some embodiments, the first locking portion or post member 72 may include an elongated proximal portion 96, and a pair of elongate legs coupled to and extending distally from a transverse distal portion (e.g., T-bar) or other coupling element at a distal end of the elongated proximal portion 96. In at least some embodiments, the transverse distal portion may be integrally formed with the elongated proximal portion 96. In some embodiments, the first locking portion or post member 72 may be formed as a single unitary structure, wherein the elongated proximal portion 96, the transverse distal portion, and/or the pair of elongate legs are integrally formed with each other and/or from a single piece of material. In some embodiments, the pair of elongate legs may secure two of the plurality of valve leaflets 22 together to form the replacement heart valve commissure assembly. Other configurations are also contemplated, and in some embodiments, the pair of elongate legs is not necessarily required to form a replacement heart valve commissure assembly or the first locking portion or post member 72.

In some embodiments, the elongated proximal portion 96 of the first locking portion or post member 72 may include a longitudinally-oriented passageway extending at least partially through the elongated proximal portion 96 of the first locking portion or post member 72, wherein the distal portion of the actuator element 15 is configured to slidably engage the longitudinally-oriented passageway of the elongated proximal portion 96 of the first locking portion or post member 72. In some embodiments, the longitudinally-oriented passageway may extend completely through the elongated proximal portion 96 of the first locking portion or post member 72. In some embodiments, a longitudinal axis of the longitudinally-oriented passageway and/or the elongated proximal portion 96 of the first locking portion or post member 72 may be arranged generally parallel to the central longitudinal axis of the expandable anchor member 17 and/or the replacement heart valve implant 16.

The longitudinally-oriented passageway may be configured to slidably receive the distal portion of the actuator element 15. The longitudinally-oriented passageway may include an internal cross-sectional shape or profile corresponding to an external cross-sectional shape or profile of the distal portion of the actuator element 15. In some embodiments, the distal portion of the actuator element 15 may be slidably disposed within the longitudinally-oriented passageway and/or may be releasably coupled to the first locking portion or post member 72 by a pinless securement feature, for example. In addition or alternatively, in some embodiments, the distal portion of the actuator element 15 may be releasably coupled to the first locking portion or post member 72 by a removable locking element, such as a pin. In some embodiments, the elongated proximal portion 96 may include at least one aperture extending through a wall of the elongated proximal portion and into the longitudinally-oriented passageway, wherein the at least one aperture is configured to engage the pinless securement feature of the distal portion of the actuator element 15. In some embodiments, at least a portion of the distal portion of the actuator element 15 may extend into the longitudinally-oriented passageway when the distal portion of the actuator element 15 is engaged with the longitudinally-oriented passageway of the elongated proximal portion 96 of the first locking portion or post member 72, for example in the elongated “delivery” configuration and/or the “everted” configuration.

In some embodiments, the distal portion of the actuator element 15 may include the pinless securement feature. The pinless securement feature does not require the distal portion of the actuator element 15 to be directly secured to the elongated proximal portion 96 of the first locking portion or post member 72 by a separate locking pin or other securing member, in order to secure the replacement heart valve implant 16 to the delivery device 11. Some examples of a pinless securement feature may include at least one projection configured to extend into the at least one aperture of the elongated proximal portion 96 of the first locking portion or post member 72, or at least one flexible leg configured to extend into the at least one aperture of the elongated proximal portion 96 of the first locking portion or post member 72. The at least one projection and/or the at least one flexible leg may be configured to deflect (e.g., radially, circumferentially, etc.) to disengage the actuator element 15 from the first locking portion or post member 72. In some embodiments, the pinless securement feature may include a threaded feature configured to rotatable engage mating threads formed in and/or on the elongated proximal portion 96 of the first locking portion or post member 72.

In some embodiments, the first locking portion or post member 72 may be disposed within the lumen of the replacement heart valve implant 16 and/or the expandable anchor member 17 proximate the distal or inflow end of the replacement heart valve implant 16 and/or the expandable anchor member 17 when the expandable anchor member 17 is in the elongated “delivery” configuration and/or the “everted” configuration. In some embodiments, at least a portion of the first locking portion or post member 72 may be disposed distal of the expandable anchor member 17 when the expandable anchor member 17 is in the elongated “delivery” configuration and/or the “everted” configuration.

In some embodiments, a first leg of the first locking portion or post member 72 and a second leg of the first locking portion or post member 72 may be laterally and/or circumferentially spaced apart from each other to define a longitudinally-oriented tissue slot extending through the first locking portion or post member 72 in a radial direction relative to the central longitudinal axis of the replacement heart valve implant 16 and/or the expandable anchor member 17. In some embodiments, a length of the longitudinally-oriented tissue slot may extend and/or may be oriented generally longitudinally with respect to the expandable anchor member 17 and/or the replacement heart valve implant 16.

In some embodiments, the elongated proximal portion 96 of the first locking portion or post member 72 may include a transversely-oriented depression and/or ridge 100 proximate a proximal end of the elongated proximal portion 96. As will be explained further below, the transversely-oriented depression and/or ridge 100 of the elongated proximal portion 96 may be configured to engage a transversely-oriented ridge of the second locking portion or buckle member 74 to lock the replacement heart valve implant 16 and/or the expandable anchor member 17 in the “deployed” configuration.

In some embodiments, the elongated proximal portion 96 of the first locking portion or post member 72 may include a keying or orienting shape formed in and/or extending longitudinally along a length and/or an outer surface of the elongated proximal portion 96 of the first locking portion or post member 72. In some embodiments, the keying or orienting shape may extend along an entire length of the elongated proximal portion 96 of the first locking portion or post member 72. The keying or orienting shape may serve as an alignment and/or anti-rotation feature with respect to the second locking portion or buckle member 74. For example, the keying or orienting shape may prevent relative rotation between the first locking portion or post member 72 and the second locking portion or buckle member 74 when the elongated proximal portion 96 of the first locking portion or post member 72 is engaged with the second locking portion or buckle member 74. Some suitable but non-limiting materials for the first locking portion or post member 72 and/or the elongated proximal portion 96, for example metallic materials or polymeric materials, are described below.

The second locking portion or buckle member 74 may include a base portion having a longitudinal axis extending between a proximal end and a distal end of the second locking portion or buckle member 74. The second locking portion or buckle member 74 may include a body portion fixedly attached to and/or integrally formed with the base portion, the body portion defining a longitudinal channel extending through the body portion of the second locking portion or buckle member 74. In at least some embodiments, the longitudinal channel may be oriented substantially parallel with the longitudinal axis of the base portion and/or the central longitudinal axis of the replacement heart valve implant 16 and/or the expandable anchor member 17. In some embodiments, at least a part of the body portion may extend away from a distal portion of a top surface of the base portion. For example, the body portion may extend radially inward from the base portion relative to the central longitudinal axis of the replacement heart valve implant 16 and/or the expandable anchor member 17.

In some embodiments, the body portion of the second locking portion or buckle member 74 may include a flap portion 98 extending proximally and/or toward the proximal end of the base portion from the body portion. In some embodiments, the flap portion 98 may include a transversely-oriented ridge extending toward the base portion and laterally across the base portion, such that when the second locking portion or buckle member 74 is viewed along the longitudinal axis of the base portion, the transversely-oriented ridge obstructs at least a portion of the longitudinal channel. In some embodiments, the body portion and/or the flap portion 98 of the second locking portion or buckle member 74 may include at least one hole or aperture formed therein for attaching a radiopaque marker to the second locking portion or buckle member 74 to aid in visualization of the second locking portion or buckle member 74.

The flap portion 98 may be configured to deflect radially relative to the central longitudinal axis of the expandable anchor member 17 and/or the replacement heart valve implant 16. The ramp 102 of the actuator element 15 may be configured to deflect the flap portion 98 of the second locking portion or buckle member 74 radially inward (e.g., toward the central longitudinal axis) as the ramp (and the first locking portion or post member 72 engaged thereto) is longitudinally translated through the longitudinal channel of the body portion of the second locking portion or buckle member 74. In some embodiments, the flap portion 98 may be biased or self-biased toward a neutral position aligned with the body portion and/or may be biased or self-biased into the longitudinal channel and/or toward the base portion of the second locking portion or buckle member 74.

In some embodiments, the second locking portion or buckle member 74 may include the projecting portion 108 (e.g., FIG. 8) at a proximal end of the base portion of the second locking portion or buckle member 74, the projecting portion 108 being configured to releasably attach the replacement heart valve implant 16 to the delivery device 11 and/or the plurality of fingers 78 of the coupler 76 via the collar 80. In at least some embodiments, the longitudinal channel may have a keyed, directional, or non-round cross-sectional profile or shape configured to slidably receive the elongated proximal portion 96 of the first locking portion or post member 72. The first locking portion or post member 72 may have an external cross-sectional profile or shape corresponding to the keyed, directional, or non-round internal cross-sectional profile or shape of the longitudinal channel. As such, the first locking portion or post member 72 may be non-rotatable relative to the second locking portion or buckle member 74 when the elongated proximal portion 96 of the first locking portion or post member 72 is engaged with and/or at least partially disposed within the longitudinal channel of the second locking portion or buckle member 74. Some suitable but non-limiting materials for the second locking portion or buckle member 74, for example metallic materials or polymeric materials, are described below.

During delivery, the replacement heart valve implant 16 may be releasably attached to the distal end of the coupler 76 and/or the inner shaft 14 by two elongated tines 104 (e.g., FIG. 8) of the finger 78 of the coupler 76 being matingly coupled with the projecting portion 108 of the second locking portion or buckle member 74 by the collar 80, and by the actuator element 15 being releasably coupled to its corresponding first locking portion or post member 72, for example by the pinless securement feature. The collar 80 may include a stop element configured to selectively prevent disengagement of the collar 80 from the locking mechanism 70 and/or the second locking portion or buckle member 74. In at least some embodiments, the stop element may include a leg portion 82 of the collar 80 that is deflectable in a circumferential direction relative to the central longitudinal axis (e.g., is a direction circling around the central longitudinal axis). The leg portion 82 of the collar 80 may extend longitudinally along a side of the collar 80. At the collar 80, the locking rod 79 may be circumferentially offset from the finger 78 of the coupler 76 with respect to the central longitudinal axis. In the latch position, shown in FIGS. 3-5 for example, the locking rod 79 may engage the stop element and/or the leg portion 82 of the collar 80. In the latch position, at least a portion of the locking rod 79 distal of a proximal end of the collar 80 may be disposed outside of at least a portion of the collar 80. For example, at least a portion of the locking rod 79 distal of the proximal end of the collar 80 may be located exterior to an outer surface of the collar 80 and/or is not disposed within the collar 80.

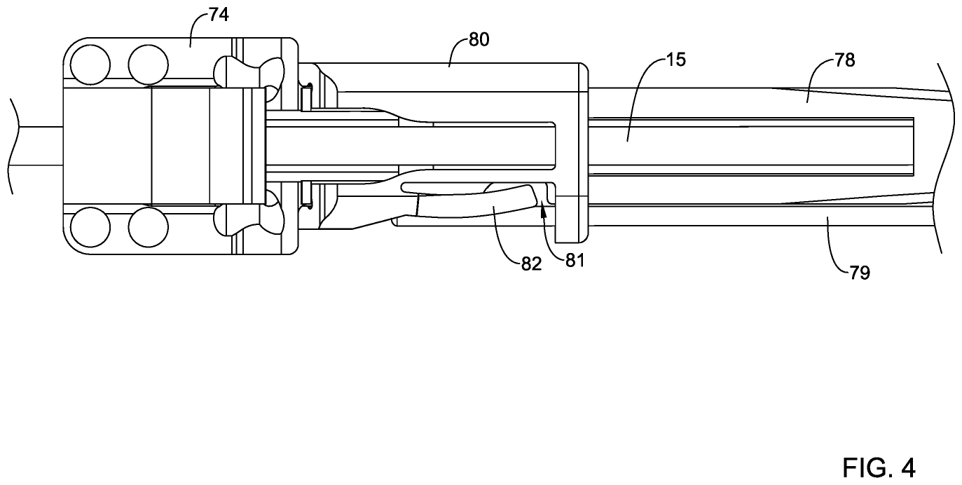

In the latch position, the locking rod 79 may secure the collar 80 in the first position. In at least some embodiments, in the first position, the collar 80 may be disposed immediately adjacent and/or in contact with the locking mechanism 70 and/or the second locking portion or buckle member 74. In some embodiments, in the latch position, the locking rod 79 may deflect the leg portion 82 of the collar 80 inward toward the finger 78 of the coupler 76. In some embodiments, the leg portion 82 of the collar 80 may include a longitudinally-oriented slot 84 configured to receive at least a portion of the finger 78 of the coupler 76 when the locking rod 79 is in the latch position. In some embodiments, in the latch position, the locking rod 79 deflects a proximal end of the leg portion 82 of the collar 80 into a recess 81 formed in the finger 78 of the coupler 76, as shown in FIG. 4 for example, thereby preventing the collar 80 from sliding from the first position to the second position. The recess 81 formed in the finger 78 of the coupler 76 may define an engagement surface facing at least partially in a distal direction and/or toward a proximal end of the leg portion 82 of the collar 80 deflected into the recess 81. When the locking rod 79 is in the latch position, interference between the engagement surface and the proximal end of the leg portion 82 of the collar 80 may prevent the collar 80 from sliding from the first position to the second position. When the leg portion 82 of the collar 80 is deflected into the recess 81, the leg portion 82 of the collar 80 may be biased or self-biased outwardly from the finger 78 of the coupler 76 toward a neutral configuration. In at least some embodiments, the leg portion 82 of the collar 80 may be configured and/or self-biased to be in the neutral configuration (e.g., FIG. 7). In some embodiments, the actuator element 15 may extend through the collar 80 and the second locking portion or buckle member 74 of the locking mechanism 70.

When the replacement heart valve implant 16 is advanced within the anatomy to the area of interest, the outer sheath 12 may be translated and/or actuated proximally to expose the replacement heart valve implant 16. As can be appreciated, a proximal end of the first locking portion or post member 72 and a distal end of the second locking portion or buckle member 74 may be longitudinally separated and/or spaced apart in the “delivery” configuration, as seen in FIG. 3 for example. In at least some embodiments, the first locking portion or post member 72 may be longitudinally actuatable and/or translatable relative to the second locking portion or buckle member 74 in the “delivery” configuration, and/or between the “delivery” configuration and the “deployed” configuration.

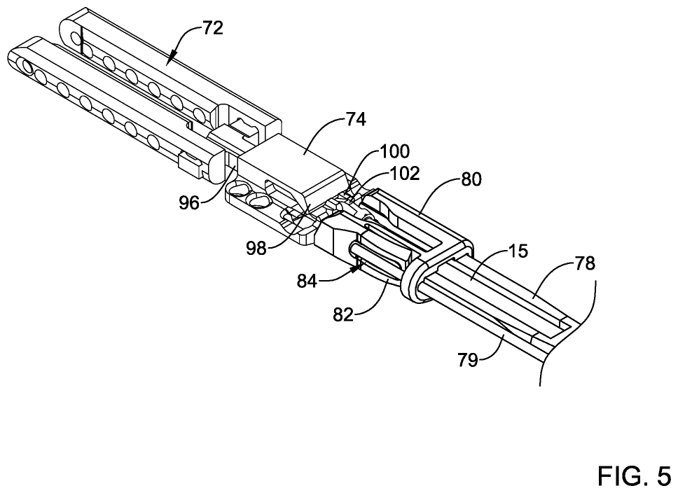

Then, the actuator element 15 can be actuated (e.g., proximally retracted) to axially shorten and/or radially expand the replacement heart valve implant 16 and/or the expandable anchor member 17 from the “delivery” configuration toward the “deployed” configuration by proximally retracting and/or translating the actuator element 15 to pull the first locking portion or post member 72 into engagement with the second locking portion or buckle member 74, as seen in FIG. 5, using the handle 18, the control knob 19, and/or the mechanism disposed within the handle 18 responsive to the control knob 19 for example.

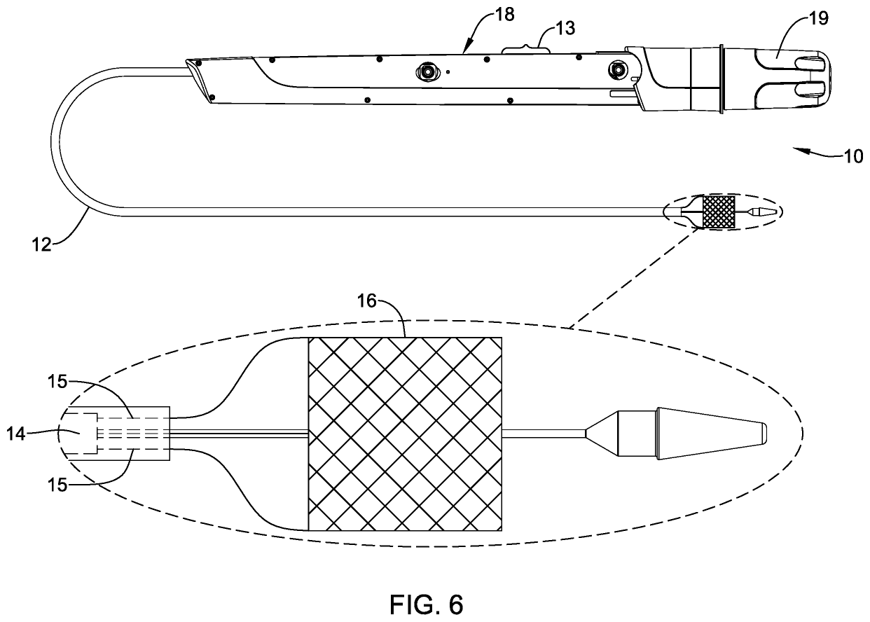

As the first locking portion or post member 72 is actuated and/or translated in a first (e.g., proximal) direction through and/or relative to the second locking portion or buckle member 74, the transversely-oriented depression engages the transversely-oriented ridge of the flap portion 98 of the second locking portion or buckle member 74 to lock the expandable anchor member 17 and/or the replacement heart valve implant 16 into the “deployed” configuration, as seen in FIGS. 5 and 6. Engagement of the transversely-oriented depression with the transversely-oriented ridge of the flap portion 98 of the second locking portion or buckle member 74 may limit or prevent distal movement and/or axial translation of the first locking portion or post member 72 relative to the second locking portion or buckle member 50 in the “deployed” configuration after the actuator element 15 has been disengaged from the locking mechanism 70 and/or the first locking portion or post member 72. Following locking of the expandable anchor member 17 and/or the replacement heart valve implant 16 in the “deployed” configuration, positioning of the replacement heart valve implant 16 may be verified using a suitable imaging technique. In the “deployed” configuration, the locking rod 79 is disposed in the latch position, and the collar 80 is disposed in the first position. As such, the replacement heart valve implant 16 and/or the expandable anchor member 17 remains releasably attached to the coupler 76 and/or the delivery device 11.

In some embodiments and/or some procedures, it may be desirable to remove and/or reposition the replacement heart valve implant 16 and/or the expandable anchor member 17. To do so, a clinician may urge and/or translate the actuator element 15 in a second (e.g., distal) direction to extend and/or elongate the expandable anchor member 17 back towards the “delivery” configuration. Axial translation of the actuator element 15 in the second (e.g., distal) direction relative to the locking mechanism 70 (e.g., the first locking portion or post member 72 and/or the second locking portion or buckle member 74) may slidably engage the ramp 102 of the actuator element 15 with the flap portion 98 and/or the transversely-oriented ridge of the flap portion 98 the second locking portion or buckle member 74, thereby deflecting the flap portion 98 of the second locking portion or buckle member 74 away from the longitudinal channel of the second locking portion or buckle member 74 and/or the actuator element 15 and/or radially inward relative to the central longitudinal axis of the expandable anchor member 17, and permitting the first locking portion or post member 72 to pass back through and/or out of the longitudinal channel of the second locking portion or buckle member 74, thereby shifting the replacement heart valve implant 16 back towards the “delivery” configuration. The replacement heart valve implant 16 and/or the expandable anchor member 17 may be repositioned and again actuated from the “delivery” configuration toward the “deployed” configuration, and placement verified using an appropriate imaging technique.

After verifying satisfactory placement of the replacement heart valve implant 16, such as by an appropriate imaging technique, the locking rod 79 may be actuated from the latch position to the release position, as seen in FIG. 7, using the handle 18, the control knob 19, the mechanism disposed within the handle 18 responsive to the control knob 19, and/or the slider 13. The release position may be disposed proximal of the latch position. At any point prior to the actuation of the locking rod 79 from the latch position to the release position, the replacement heart valve implant 16 and/or the expandable anchor member 17 may be repositioned and/or redeployed as discussed above. When the locking rod 79 is in the release position, the stop element and/or the leg portion 82 of the collar 80 may be configured and/or self-biased to be in the neutral configuration, shown in FIG. 7. In at least some embodiments, the locking rod 79 is actuatable from the latch position to the release position, and the locking rod 79 is not actuatable from the release position to the latch position. As may be seen from FIG. 7, when the locking rod 79 is the release position and the stop element and/or the leg portion 82 of the collar 80 is in the neutral configuration, the proximal end of the stop element and/or the leg portion 82 of the collar 80 prevents distal actuation of the locking rod 79 relative to the collar 80 and/or the leg portion 82. When the locking rod 79 is in the release position, the collar 80 may be permitted to slide from the first position to the second position.

Next, when the locking rod 79 is in the release position, the distal portion of the actuator element 15 may be permitted to be pulled in the first (e.g., proximal) direction out of the first locking portion or post member 72 by rotation of the control knob 19 and/or the mechanism disposed within the handle 18 responsive to the control knob 19. Proximal translation of the actuator element 15 causes the ramp 102 to subsequently engage the collar 80 and thereby retract the collar 80 from the two elongated tines 104 and the projecting portion 108, shifting the collar 80 from the first position to the second position, shown in FIG. 8. In at least some embodiments, the second position may be proximal of the first position.

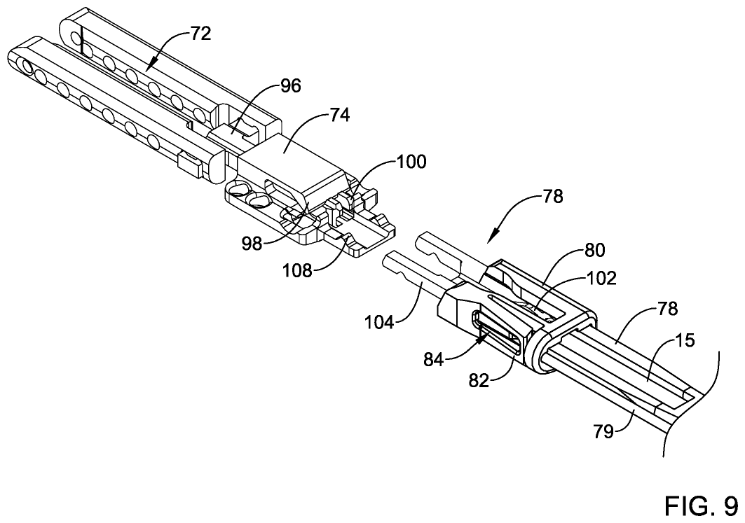

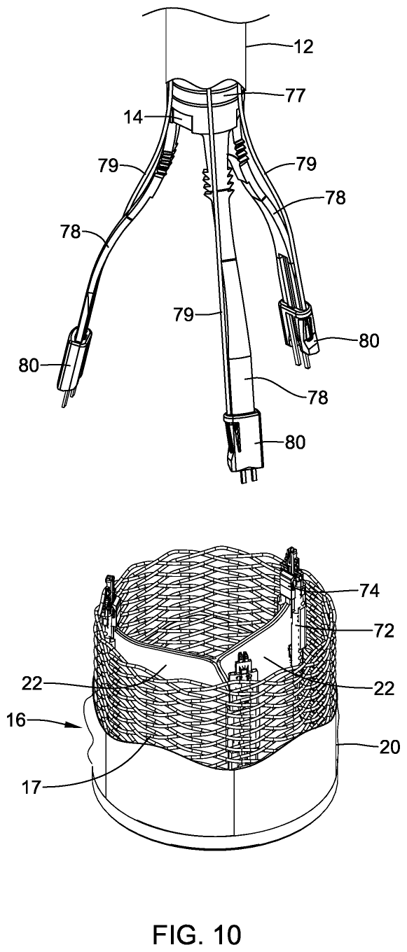

Once the locking rod 79 has been actuated to the release position, the stop element and/or the leg portion 82 has shifted to the neutral configuration, and the actuator element 15 proximally translated out of the first locking portion or post member 72, the replacement heart valve implant 16 may not be repositionable. After the collar 80 has been retracted to the second position, the two elongated tines 104 may decouple from the projecting portion 108, as seen in FIG. 9, and the finger 78 of the coupler 76 may be withdrawn from the replacement heart valve implant 16 thereby leaving the replacement heart valve implant 16 (and/or the expandable anchor member 17) in the anatomy at the area of interest in a “released” configuration, as seen in FIG. 10.

The materials that can be used for the various components of the medical device system 10, the delivery device 11, the outer sheath 12, the inner shaft 14, the replacement heart valve implant 16, the handle 18, etc. (and/or other systems disclosed herein) and the various elements thereof disclosed herein may include those commonly associated with medical devices. For simplicity purposes, the following discussion makes reference to the medical device system 10, the delivery device 11, the outer sheath 12, the inner shaft 14, the replacement heart valve implant 16, the handle 18, etc. However, this is not intended to limit the devices and methods described herein, as the discussion may be applied to other elements, members, components, or devices disclosed herein, such as, but not limited to, the slider 13, the actuator element(s) 15, the expandable anchor member 17, the control knob 19, the seal member 20, the plurality of valve leaflets 22, the first locking portion or post member 72, the second locking portion or buckle member 74, the coupler 76, the finger 78, the locking rod 79, the collar 80, etc., and/or elements or components thereof.

In some embodiments, the medical device system 10, the delivery device 11, the outer sheath 12, the inner shaft 14, the replacement heart valve implant 16, the handle 18, etc., and/or components thereof (such as, but not limited to, the slider 13, the actuator element(s) 15, the expandable anchor member 17, the control knob 19, the seal member 20, the plurality of valve leaflets 22, the first locking portion or post member 72, the second locking portion or buckle member 74, the coupler 76, the finger 78, the locking rod 79, the collar 80, etc.), may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical device system 10, the delivery device 11, the outer sheath 12, the inner shaft 14, the replacement heart valve implant 16, the handle 18, etc., and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device system 10, the delivery device 11, the outer sheath 12, the inner shaft 14, the replacement heart valve implant 16, the handle 18, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 10, the delivery device 11, the outer sheath 12, the inner shaft 14, the replacement heart valve implant 16, the handle 18, etc. to achieve the same result.

In some embodiments, a degree of Magnetic Resonance Imaging (MRI) compatibility is imparted into the medical device system 10, the delivery device 11, the outer sheath 12, the inner shaft 14, the replacement heart valve implant 16, the handle 18, etc. For example, the medical device system 10, the delivery device 11, the outer sheath 12, the inner shaft 14, the replacement heart valve implant 16, the handle 18, etc., and/or components or portions thereof, may be made of a material that does not substantially distort the image and create substantial artifacts (e.g., gaps in the image). Certain ferromagnetic materials, for example, may not be suitable because they may create artifacts in an MRI image. The medical device system 10, the delivery device 11, the outer sheath 12, the inner shaft 14, the replacement heart valve implant 16, the handle 18, etc., or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the medical device system 10, the delivery device 11, the outer sheath 12, the inner shaft 14, the replacement heart valve implant 16, the handle 18, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the medical device system 10, the outer sheath 12, the inner shaft 14, the replacement heart valve implant 16, the handle 18, the seal member 20, etc. and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the medical device system 10, the outer sheath 12, the inner shaft 14, the replacement heart valve implant 16, the handle 18, the seal member 20, etc. may include and/or be formed from a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, the medical device system 10, the delivery device 11, the outer sheath 12, the inner shaft 14, the replacement heart valve implant 16, the handle 18, etc. may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made to details, particularly in matters of shape, size, and arrangement of steps, without exceeding the scope of the invention. This may include, to the extent that it is appropriate, the use of any of the features of one example embodiment being used in other embodiments. The invention's scope is, of course, defined in the language in which the appended claims are expressed.

What is claimed:

1-15. (canceled)

16. A medical device system, comprising:
a delivery device including an outer sheath and an inner shaft having a coupler fixed to a distal end of the inner shaft; and
a replacement heart valve implant releasably attached to the coupler, the replacement heart valve implant including:
an expandable anchor member defining a central longitudinal axis; and
a locking mechanism attached to the expandable anchor member, the locking mechanism being configured to engage with the coupler;
wherein the delivery device includes a collar configured to releasably secure the coupler to the locking mechanism;
wherein the collar includes a stop element configured to selectively prevent disengagement of the collar from the locking mechanism.

17. The medical device system of claim 16, wherein the collar is slidable over the coupler between a first position and a second position;
wherein in the first position the collar secures the coupler to the locking mechanism, and in the second position the coupler is disengageable from the locking mechanism; and
the delivery device further including a locking rod extending through at least a portion of the collar, the locking rod being actuatable between a latch position and a release position;
wherein in the latch position the locking rod engages the stop element.

18. The medical device system of claim 17, wherein the stop element includes a leg portion deflectable in a circumferential direction relative to the central longitudinal axis.

19. The medical device system of claim 18, wherein the leg portion includes a longitudinally-oriented slot configured to receive at least a portion of the coupler when the locking rod is in the latch position.

20. The medical device system of claim 18, wherein the leg portion extends longitudinally along a side of the collar.

21. The medical device system of claim 17, wherein the second position is proximal of the first position.

22. The medical device system of claim 17, wherein the release position is proximal of the latch position.

23. The medical device system of claim 17, wherein the locking rod is actuatable from the latch position to the release position and the locking rod is not actuatable from the release position to the latch position.

24. A medical device system, comprising:
a delivery device including an outer sheath and an inner shaft having a coupler fixed to a distal end of the inner shaft; and
a replacement heart valve implant releasably attached to the coupler, the replacement heart valve implant including:
an expandable anchor member defining a central longitudinal axis; and
a locking mechanism attached to the expandable anchor member, the locking mechanism being configured to engage with the coupler;
wherein the delivery device includes a collar slidable over the coupler between a first position and a second position, the collar being configured to releasably secure the coupler to the locking mechanism;
wherein in the first position the collar secures the coupler to the locking mechanism, and in the second position the coupler is disengageable from the locking mechanism; and
the delivery device further including a locking rod extending through at least a portion of the collar, the locking rod being actuatable between a latch position and a release position;
wherein in the latch position the locking rod secures the collar in the first position.

25. The medical device system of claim 24, wherein in the latch position a portion of the locking rod distal of a proximal end of the collar is disposed outside of at least a portion of the collar.

26. The medical device system of claim 24, wherein in the latch position the locking rod deflects a leg portion of the collar inward toward the coupler.

27. The medical device system of claim 26, wherein in the latch position the locking rod deflects a proximal end of the leg portion of the collar into a recess formed in the coupler.

28. The medical device system of claim 24, wherein when the locking rod is in the release position the collar is permitted to slide from the first position to the second position.

29. The medical device system of claim 28, wherein the delivery device includes an actuator element extending through the collar and releasably engaged with the locking mechanism.

30. The medical device system of claim 29, wherein when the locking rod is in the release position proximal translation of the actuator element shifts the collar from the first position to the second position.

31. A medical device system, comprising:
a delivery device including an outer sheath and an inner shaft having a coupler fixed to a distal end of the inner shaft, the outer sheath and the inner shaft extending distally from a handle; and
a replacement heart valve implant releasably attached to the coupler, the replacement heart valve implant including:
an expandable anchor member defining a central longitudinal axis; and
a locking mechanism attached to the expandable anchor member, the locking mechanism being configured to engage with the coupler;
wherein the locking mechanism includes a buckle member fixedly attached to the expandable anchor member, and a post member coupled to the expandable anchor member distal of the buckle member;
wherein the delivery device includes a collar slidable over the coupler between a first position and a second position;
wherein in the first position the collar secures the coupler to the buckle member, and in the second position the coupler is disengageable from the buckle member; and
the delivery device further including a locking rod extending through at least a portion of the collar, the locking rod being actuatable between a latch position and a release position;
wherein in the latch position the locking rod deflects a leg portion of the collar into a recess formed in the coupler, thereby preventing the collar from sliding from the first position to the second position.

32. The medical device system of claim 31, wherein the leg portion is self-biased to be in a neutral configuration.

33. The medical device system of claim 32, wherein when the leg portion is deflected into the recess, the leg portion is self-biased outwardly from the coupler.

34. The medical device system of claim 31, wherein the recess defines an engagement surface facing at least partially in a distal direction;
wherein when the locking rod is in the latch position, interference between the engagement surface and a proximal end of the leg portion prevents the collar from sliding from the first position to the second position.

35. The medical device system of claim 31, wherein at the collar, the locking rod is circumferentially offset from the coupler with respect to the central longitudinal axis.
